(19) United States
(12) Patent Application Publication
(10) Pub. No.: US 2023/0358721 A1
(43) Pub. Date: Nov. 9, 2023

(54) METHOD FOR ANALYZING THE COMPOSITION OF HYDROCARBONS BY MEANS OF A PYROLYSER WITHOUT SEPARATION DEVICE

(71) Applicant: VINCI TECHNOLOGIES SAS, Nanterre (FR)

(72) Inventors: Jérémie AMMOUIAL, Boulogne-Billancourt (FR); Nicolas BOUTON, Villejuif (FR)

(73) Assignee: VINCI TECHNOLOGIES SAS, Nanterre (FR)

(21) Appl. No.: 18/312,829

(22) Filed: May 5, 2023

(30) Foreign Application Priority Data
May 9, 2022 (FR) 2204374

(51) Int. Cl. G01N 33/24; G01N 31/12; G01N 33/28

(52) U.S. Cl. CPC G01N 33/241; G01N 31/12; G01N 33/2823

(57) ABSTRACT

The invention relates to a method for the continuous characterization and quantification of C1-C120 hydrocarbons present in a single sample taken from a sedimentary basin, said method including pyrolysis under a non-oxidizing atmosphere of the sample, the entrainment of the gaseous pyrolysis effluents by means of an inert gas stream and then the analysis of said effluents in order to obtain their profiles as a function of the pyrolysis temperature, wherein said profiles are deconvolved into sub-areas corresponding to each class of isomer via a reference chart established for each of said classes and obtained using the empirical Antoine equation derived from the integration of the Clausius-Clapeyron physical law for an assumed constant enthalpy of vaporization;

### CROSS-REFERENCE TO RELATED APPLICATIONS

This application claims the benefit of priority under 35 U.S.C. 119 to French Patent Application No. 2204374, filed May 9, 2022; the disclosure of which is incorporated herein by reference in its entirety.

### FIELD

The present invention relates to a method for the continuous characterization of a composition of oils and crude oils that can be analyzed in a free state or while being contained in a sedimentary rock.

### BACKGROUND

The parameters sought by the method of the invention comprise: the composition in terms of hydrocarbons, the average molecular weight of the sample and the boiling point (known as the “true boiling point”).

The applications targeted by the invention are primarily in sectors of the oil industry, in particular in the exploration phase and in the refining phase. More specifically, the invention relates to determining, during the first exploration studies on the scale of the sedimentary basin, the composition of the hydrocarbons contained in source rocks or in reservoir rocks, from rock debris (or “cuttings”) dislodged by the drilling tool and brought to the surface by the drilling fluid.

The geological nature of such debris, its physical characteristics and its content in various fluids (in particular water, hydrocarbons and other gases, etc.) can provide valuable information about rocks drilled without the need to extract these fluids from the samples collected, in a reduced time and automatically. However, the invention may also relate to other fields of application. such as the analysis of sites and soils that have been polluted by hydrocarbons.

Oil originates from the kerogen contained in source rocks and from the organic matter present in the environment at the moment of sedimentation of said source rock (SR). Depending on the depositional environment of this source rock, the resulting kerogen may be of three different types. Type I corresponds to a lacustrine environment in which the organic matter produced is enriched in lipids. Type II corresponds to a marine-type depositional environment in which the precursor organic matter is composed of both lipids and cellulose. Type III organic matter, on the other hand, corresponds to a continental-type depositional environment with original organic matter enriched in cellulose and lignin. Depending on the type of kerogen, the composition of the oil created by thermal cracking of the kerogen, during the burial of the source rock over geological time, will vary. The quality of the oil is then directly affected.

The progress rate of the thermal cracking of the kerogen considered is also a fundamental parameter to be taken into consideration when studying the composition of an oil. In the early stages of the thermal cracking of kerogen, the oil obtained is heavy (i.e., composed of numerous hydrocarbons with long carbon chains), which corresponds to the entry of the source rock into the oil window (temperature window corresponding to a generation of liquid hydrocarbons from kerogen). Subsequently, as the depth of the source rock increases due to the phenomenon of subsidence, thermal cracking produces hydrocarbons with increasingly short chains from the kerogen until reaching the dry gas window in which the only hydrocarbon produced is the smallest, i.e., methane. Secondly, heavy hydrocarbons, whether or not they have migrated from the source rock, can undergo a second thermal cracking called secondary cracking if the surrounding temperature is sufficiently high.

The last important factor explaining the quality of the oil considered is its preservation over geological time. If the hydrocarbons are brought close to the surface, their quality can be reduced due to archaea-type organisms able to live at temperatures above 100° C. and feeding on n-alkanes. The direct consequence is a drastic decrease in the API degree of the oil (increase in its density) as well as an enrichment in molecules having heteroatoms comprising nitrogen, sulfur and oxygen (NSO).

All of these parameters explain the great variability in the composition of oils present on Earth. Thus, the invention will provide users with essential data from the start of exploratory drilling about the composition of the oil present on the prospected site.

The Rock-Eval technique (registered trademark—IFP Energies nouvelles) is known, described in particular in patents FR2472754 and EP0691540B1. This analysis technique, which is fast and almost automatic, was developed for characterizing organic matter and hydrocarbons contained in a rock sample from a geological formation. More specifically, this technique makes it possible to determine the presence, nature and degree of maturity of the organic matter contained in a rock sample.

The Rock-Eval method also provides precise information on the quantification of hydrocarbons, the quantity of total organic carbon (TOC) and the quantity of mineral carbon (MinC) contained in a rock sample. Thus, the Rock-Eval technique makes it possible in particular to measure the quantity of hydrocarbon compounds released throughout pyrolysis. It is then possible to establish a pyrogram which is a curve representing the evolution of the quantity of hydrocarbon compounds released relative to the weight of the sample considered as a function of time. A pyrogram usually has several peaks. The peaks are well differentiated, and the surface area of each peak is calculated. Thus, for each peak, a magnitude representative of the quantity of hydrocarbon compounds released over a range of temperatures surrounding the peak considered is obtained.

Two main methods implementing two different temperature sequences have been developed. The “Basic method” or “Bulk Rock method”, dedicated more particularly to source rock samples, is described for example in Lafargue et al. (1998) and Behar et al. (2001). The “Reservoir method”, dedicated more particularly to reservoir rock samples, is for example described in patent EP0691540B1.

Patent EP3918321 describes a method for analyzing C1-C6 gaseous hydrocarbons by means of a multi-gas analyzer allowing for the quantification of said gaseous hydrocarbons. However, this method does not allow the quantification of hydrocarbons comprising more than 6 carbons because the transport of these hydrocarbons to the infrared analyzer is done only in the gaseous state under atmospheric pressure and temperature conditions, which does not correspond to the physical state of hydrocarbons comprising more than six carbons.

Furthermore, it has been found necessary to improve the precision of the results obtained with the Rock-Eval® method and to optimize the interpretation thereof so that the economic and technical decisions relating to oil exploration are based on more solid foundations. Since the beginnings of the implementation of the Rock-Eval method, many methods for characterizing thermo-vaporizable hydrocarbons contained in rocks have been developed but none has made it possible to precisely describe the composition of these hydrocarbons. The existing methods (disclosed previously as well as others developed more recently) make it possible at most to estimate the relative molecular weights of each hydrocarbon fraction analyzed by attempting to segment the signal from the flame ionization detectors (FID) via temperature levels determined arbitrarily and without any real physical consistency.

It is in this context that the invention has sought to develop a new method making it possible to solve the technical problems posed by the prior methods in order to quantify these liquid and solid hydrocarbons under atmospheric conditions by means of a simple and rapid method making it possible to obtain reliable and novel results in just a few steps and from a sample of small quantity and of various types.

The physical principle underlying the characterization method of the invention is based on the Antoine equation (simplification of the Clausius-Clapeyron thermodynamic relation) which describes the evolution of the saturation vapor pressure with the rise in temperature when liquid-vapor equilibrium is reached. It is this physical law which mainly occurs when the temperature is raised during the pyrolysis of a sample of rocks containing free hydrocarbons.

The analysis of a very small quantity of hydrocarbons (a few microliters at most) contained in the rocks requires the use of the Antoine equation to relate the mass of evaporated hydrocarbon (i.e., having passed from the liquid phase to the gaseous phase) to the temperature of this phase change. For example, if pure n-decane (the boiling point of which is 174.1° C.) is analyzed in pyrolysis via a constant temperature rise, the maximum evaporation temperature of n-decane will be lower than the theoretical boiling point (between 70 and 130° C. depending on the quantity of product analyzed). This observation is also accentuated by a second phenomenon occurring during the evaporation of a hydrocarbon compound, namely the diffusion of matter, which is governed by Fick's law, through an interface whose flow is directed in the opposite direction to the concentration gradient (in the direction of decreasing concentration values).

For each n-paraffin (hydrocarbons between C1 and C120), the evaporation curves relating the mass of distilled hydrocarbons to the temperature of the pyrolysis furnace were modeled using Antoine's thermodynamic relation. This approach then makes it possible to create a chart giving the position of each hydrocarbon according to its molecular weight.

Thus, the object of the invention is a method for the continuous characterization and quantification of C1-C120 hydrocarbons comprising pyrolysis under a non-oxidizing atmosphere of the sample, the entrainment of the gaseous pyrolysis effluents by means of an inert gas stream and then the analysis of said effluents in order to obtain their profiles as a function of the pyrolysis temperature, characterized in that said profiles are superimposed on the reference chart created from the Clausius-Clapeyron physical law in order to determine the composition of the hydrocarbons in the sample analyzed.

More specifically, the experimental method of the invention consists in an increase in temperature under an inert atmosphere at constant speed of said sample, from the lowest possible temperature T1 (i.e., at room temperature, even lower if it is technically feasible) to a final temperature T2 which is necessarily higher than the highest boiling point of the mixture to be analyzed by said method. Temperature T1 is therefore necessarily lower than temperature T2.

According to an advantageous feature of the method of the invention, the temperature of pyrolysis is raised with a single temperature gradient of between 0.1 and 50° C./min. Preferably, this temperature gradient is between 10 and 30° C./min.

The implementation of said method makes it possible to obtain the composition of the mixture of hydrocarbons contained in said rock sample as well as the average molar mass and the API degree via the processing of the data by the chart, wherein each of the curves of which it is composed (ranging from n-C1 to n-C120) is theoretically calculated by the following formula (F).

mᵢ=(Dᵥ×Mᵢ×t)/(R×T)×10^{A-B/(T+C)}

With xᵢ(-): molar fraction of the hydrocarbon considered
mᵢ (kg): mass of the hydrocarbon considered
Dᵥ (m³.s⁻¹): volume flow rate of the carrier gas
Mᵢ (kg.mol⁻¹): molar mass of the hydrocarbon considered
T (K): temperature
t (s): time
R (J.mol⁻¹.K⁻¹): ideal gas constant
A (Pa), B (-) and C (K): parameters of the Antoine equation
Pᵢ (Pa): partial pressure
Pₜₒₜ (Pa): total pressure of the system.

The x-axis of said chart has the temperature as its magnitude, while the y-axis has the mass as its unit. Thus, synthetically, the method according to the invention for the analysis of free oil or oil contained in a rock sample comprises a phase of pyrolysis of the sample carried out under an inert atmosphere in a range of increasing temperatures between an initial temperature T1 and a final temperature T2 according to a linear increase in temperature with kinetics of between 0.1° C./min and 50° C./min. The vaporized hydrocarbons resulting from the pyrolysis of the sample are quantified and characterized by their vaporization temperatures. The evolution of said temperatures being theoretically predicted by the Antoine equation, the hydrocarbon signal obtained can therefore be segmented into different classes of isomers from C1 to C120.

Said process may optionally be followed by an oxidizing phase in order to determine the TOC (percentage of total organic carbon) in the case of the analysis of heavy oils.

The method of the invention can integrate high-performance software which draws the curves corresponding to the profiles of the hydrocarbons, the quantitative calculations, the determination of the distributions of these hydrocarbons and which makes it possible to obtain information on the quantities of hydrocarbons obtained by pyrolysis, as well as new data on their genesis.

The implementation of the method of the invention complements and extends the detection of C1-C6 gaseous hydrocarbons which can be carried out via, for example, the method described in patent EP3918321. The minimum analysis starting temperature of 30° C. provided for in the method of the invention is not suitable for obtaining hydrocarbon analysis results below C7.

### BRIEF DESCRIPTION OF THE DRAWINGS

Other features and advantages of the method according to the invention will become apparent on reading the following description of non-limiting examples of implementation, with reference to the accompanying figures which are described below.

FIG. 1 is a schematic view of a preferred embodiment of the device for implementing the method of the invention.

FIG. 2 illustrates the temperature variation sequence according to the method of the invention.

FIG. 3 is a graph/chart created from the Antoine equation applied to the Rock-Eval pyrolysis furnace, according to a particular implementation of the method of the invention, making it possible to predict the quantity of alkane for C1 to C40 isomers.

FIG. 4 represents a superposition of the chart and a hydrocarbon curve obtained via the temperature sequence on an oil sample according to one embodiment of the method of the invention.

FIG. 5 is a superposition of the experimental curves obtained via the Rock-Eval analysis of n-C10, n-C20, n-C30 and n-C40 at different masses with theoretical simulated distillation curves of these same paraffins obtained via the Antoine equation.

FIG. 6 represents a distribution of the isomers of a crude oil and two reference oils in percentages. The black bars are the proportions obtained from the Rock-Eval simulation distillation while the hatched bars were obtained via a gas chromatography analysis.

FIG. 7 represents another distribution of the isomers of a crude oil and two reference oils in percentages. The black bars are the proportions obtained from the Rock-Eval simulation distillation while the hatched bars were obtained via a gas chromatography analysis.

FIG. 8 represents yet another distribution of the isomers of a crude oil and two reference oils in percentages. The black bars are the proportions obtained from the Rock-Eval simulation distillation while the hatched bars were obtained via a gas chromatography analysis.

FIG. 9 represents the distillation curves simulated by Rock-Eval (black solid curves) for a crude oil and for two reference oils as well as the distillation curves simulated by gas chromatography (dotted curves).

The invention generally relates to a method for characterizing hydrocarbons contained in a rock sample. This method is characterized by a linear rise in temperature under an inert atmosphere according to a slope chosen by the user and between 0.1° C. and 50° C. per minute.

### DETAILED DESCRIPTION

In particular, the invention relates to the analysis of heat-vaporizable hydrocarbon fractions and is concerned in particular with their identification by temperature. It is well known that the lightest hydrocarbons are vaporized before the heaviest. However, since the quantities of hydrocarbons analyzed are very low with the Rock-Eval method, the temperatures at which these hydrocarbons are detected do not correspond to the theoretical boiling points of the components of the sample. Since the emergence of the Rock-Eval method, which has made it possible to quantify the hydrocarbons contained in oil source rocks in a remarkable time, the determination of the composition of the thermo-vaporizable hydrocarbons contained in these rocks has become essential because this composition constitutes an essential quality criterion of the oil that these rocks contain. Estimations of the financial investment required to bring the prospective oil field into production will not be the same if the hydrocarbons present are light or heavy.

The main problem posed by determining the composition of hydrocarbon mixtures with the Rock-Eval method and the related pyrolyzers was the lack of a separation device such as, for example, during chromatographic analysis. Moreover, although the temperatures read by the Rock-Eval device are precise, they do not correspond to the actual boiling points of the compounds analyzed because these compounds are present in too low quantities.

The invention and its method of implementation described below make it possible to overcome this problem thanks to the application of the Clausius-Clapeyron law and, in particular, of the Antoine equation describing the passage of hydrocarbons from the liquid phase to the vapor phase during a rise in temperature. This method may advantageously, but not exclusively, be implemented by means of the Rock-Eval device described in patent EP2342557.

The method of the invention is applied primarily to samples of reservoir rocks, source rocks and crude oils, but can also be used for soils polluted with hydrocarbons or else for catalysts derived from refining processes. More generally, the invention may relate to any sample containing hydrocarbons, the composition of which it is necessary to know. Until now, knowledge of the composition of these hydrocarbons contained in a sample required extraction via different solvents and then chromatographic analysis. The invention is an advance in that it allows automatic, rapid and in situ hydrocarbon composition characterization.

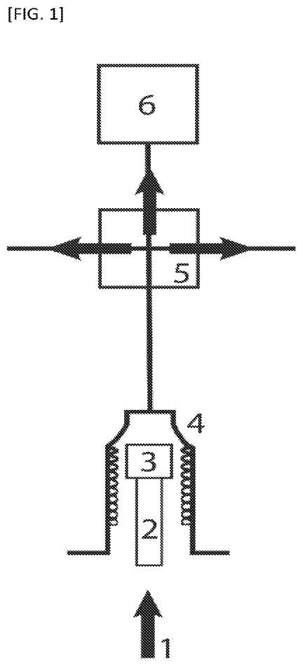

FIG. 1 schematically shows an embodiment of the device for implementing the method. This device comprises a furnace 4 intended for pyrolysis, the temperature of which can range from 0° C. to 900° C. A rock sample contained in a boat 3 is then introduced into the furnace via a piston 2. The inert carrier gas 1, continuously circulating at a constant flow rate in the system, drives the pyrolysis product towards the flame ionization detector 6 (FID), which then makes it possible to quantify the hydrocarbons. According to an alternative embodiment, the device can be provided with a heat divider 5 making it possible to redirect the flow of gas to other detectors.

FIG. 2 shows the rise in temperature T1 to temperature T2 with a single temperature gradient between 0.1 and 50° C./min. Consequently, this rise in temperature does not comprise any plateau.

The pyrolyzed hydrocarbon stream is entrained and directed by the inert gas stream, the flow rate of which may be between 20 ml/min and 70 ml/min, towards a flame ionization detector 6 (FID) (FIG. 1).

The thermo-vaporization phenomenon occurring during the pyrolysis of organic matter with thermal cracking consists of two phases. The first phase is predominant from 0° C. to 400° C. and primarily concerns relatively light hydrocarbons (below C40), whereas the second phase becomes predominant only above 400° C. and concerns in particular heavy hydrocarbons, resins, asphaltenes as well as kerogen.

Once the pyrolysis step is complete, the hydrocarbon thermo-vaporization curve is obtained. This curve can comprise one or more peaks depending on the homogeneity of the distribution of the boiling points of its compounds.

According to an advantageous embodiment, this curve can be represented by a graph of the type m=f(T) where m is the mass in mg and T is the temperature in (° K).

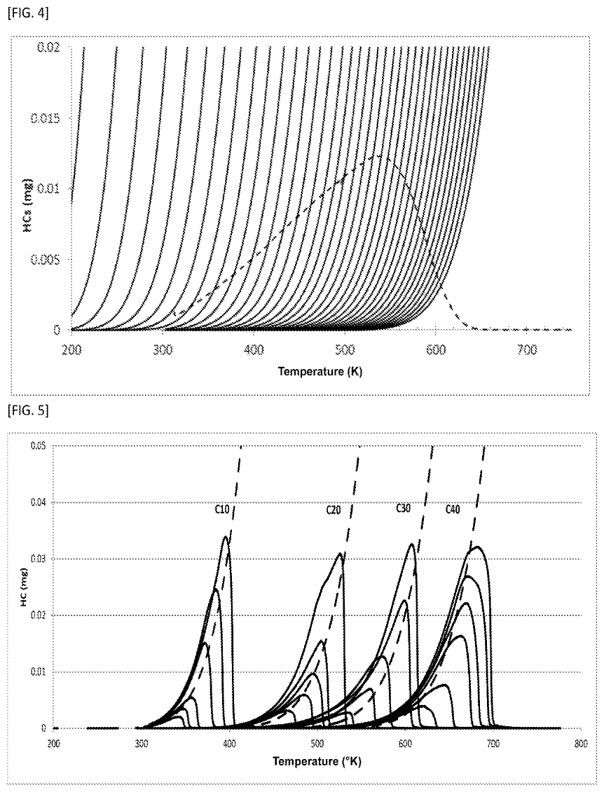

The hydrocarbon curve may then be superimposed on the theoretical vaporization chart of aliphatic n-hydrocarbons as in FIG. 4.

The component curves of the chart were determined by applying Antoine equations to pyrolysis using the Rock-Eval method. During analysis by the Rock-Eval method, the signal obtained via the FID (flame ionization detector) makes it possible to know the quantity of hydrocarbons generated during pyrolysis as a function of temperature. However, the Antoine equation makes it possible to have a relationship between the partial pressure (Pᵢ) of a gas as a function of temperature (T) in the context of a liquid-vapor equilibrium.

Log(Pᵢ)=A−(B/(T+C) wherein Pᵢ=Pₛₐₜ/Pₜₒₜ.

During the Rock-Eval analysis, equilibrium is never reached because the gas phase is constantly renewed, which constitutes the main cause of error or approximation of this method. In order to involve the mass of hydrocarbons, which is measured by the Rock-Eval method, the partial pressure Pᵢ is then expressed according to Raoult's law.

Pᵢ=xᵢ×Pₜₒₜ

After involving the ideal gas law, the following relationship (F) is obtained.

mᵢ=(Dᵥ×Mᵢ×t)/(R×T)×10^{A-B/(T+C)}

with xᵢ(-): molar fraction of the hydrocarbon considered
mᵢ (kg): mass of the hydrocarbon considered
Dᵥ (m³.s⁻¹): volume flow rate of the carrier gas
Mᵢ (kg.mol⁻¹): molar mass of the hydrocarbon considered
T (K): temperature
t (s): time
R (J.mol⁻¹.K⁻¹): ideal gas constant
A (Pa), B (-) and C (K): parameters of the Antoine equation
Pᵢ (Pa): partial pressure
Pₜₒₜ (Pa): total pressure of the system.

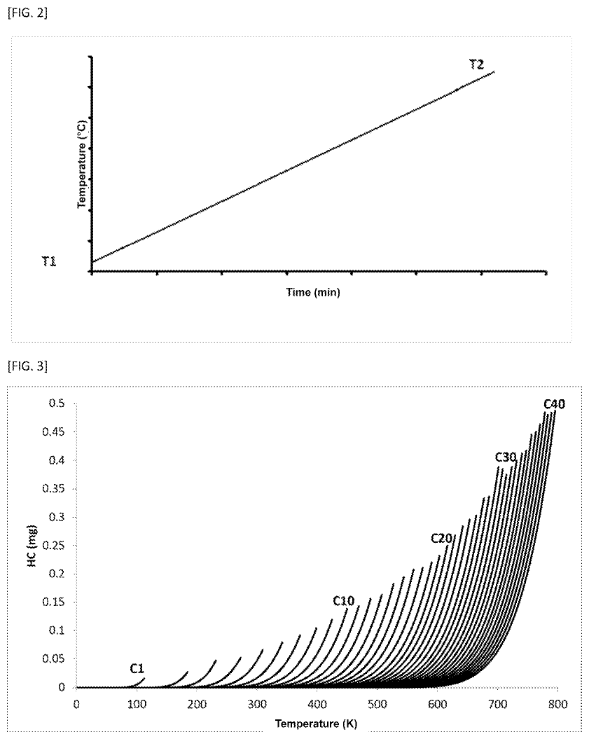

Thus, for each n-paraffin from C1 to C120, it is possible to determine the curves shown in FIG. 3 of the mass of liquid hydrocarbons passing into gaseous form as a function of temperature. The end of each curve corresponds to the theoretical boiling point of each compound. The relative position of each curve depends on the molar mass of the n-alkane and on the parameters (A-B-C) of the Antoine equation which is specific to it.

The general shape of the curves is determined by the temperature variation ramp and the volume flow rate used in the device. Thus, each curve of the chart corresponds to the evaporation of the mass of n-alkanes as a function of the increase in temperature in the pyrolysis furnace. By superposition of the hydrocarbon curve with this chart, the following approximations are therefore assumed.
The Antoine equation is used when the equilibrium between the gaseous phase and the liquid is not reached.
Each isomer behaves thermally like the corresponding n-alkane.
Each n-alkane behaves independently of the other n-alkanes within a mixture.

The hydrocarbon curve derived from the FID signal is then segmented according to an algorithm, allowing the proportion of each class of isomers within the mixture to be estimated. The algorithm used may vary depending on the use and the case studies encountered.

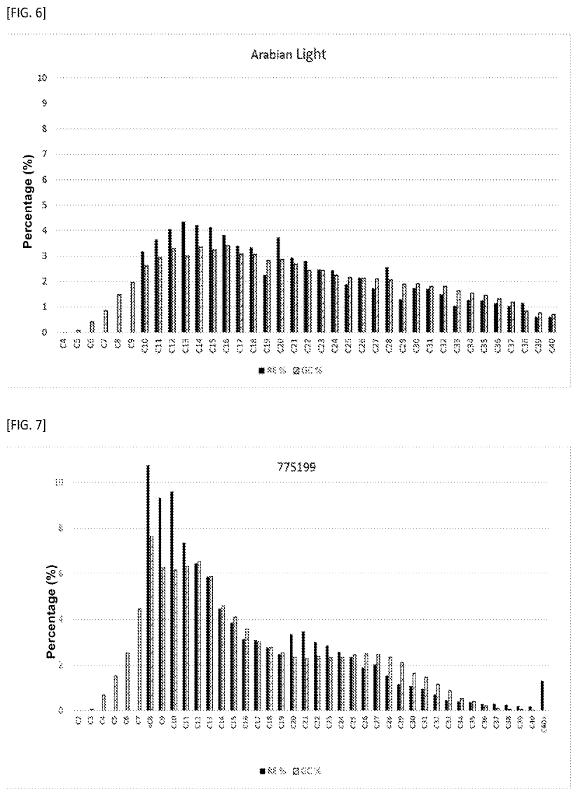

The proportions of each class of isomer obtained by the Rock-Eval RE method for oils were correlated with the proportions of the isomers obtained via gas chromatography GC (e.g., FIGS. 6 to 8). It can then be observed that the differences between the results of the two methods are very small. Although biases related to the Rock-Eval characterization method remain, they are known and controlled. They are intimately related to the approximations set out in the demonstration of the curve equations.

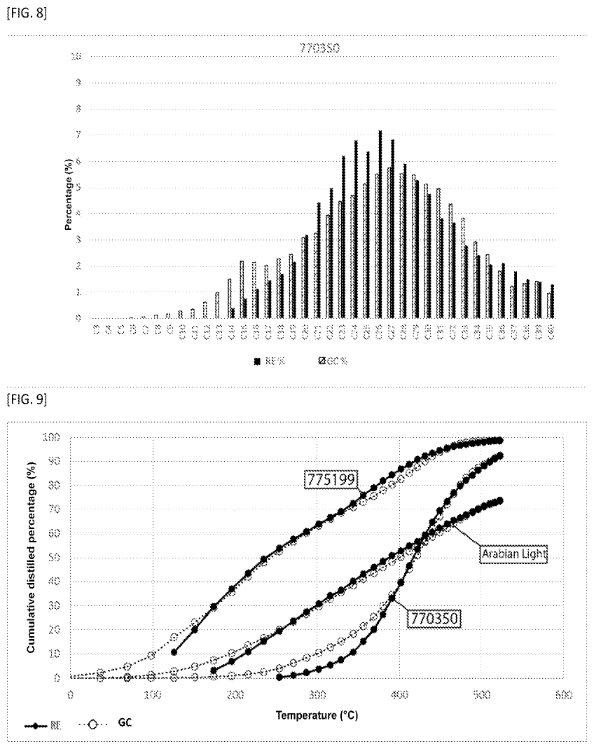

The proportions of each class of isomer then make it possible to trace simulated distillation curves. This time, the temperatures used to reconstruct these curves are the theoretical boiling points of the n-alkane of the class of isomer considered. These distillation curves simulated by Rock-Eval (solid line) are then compared with the simulated distillation curves produced by gas chromatography (dotted line) (FIG. 9).

The invention is primarily intended for the analysis of free oils contained in reservoir rocks (porous and permeable). The invention may also be used for the analysis of source rocks but may prove to be less precise in this case due to the matrix effects inherent in the argillaceous lithology of the source rocks.

In the following section, three oil samples (one crude oil and two reference oils whose boiling points are known) were analyzed using the Rock-Eval simulated distillation method and then by gas chromatography in order to compare the results.

The first oil (reference 775199) is a “light” oil because it has a high fraction of short-chain hydrocarbons. The second oil (reference 770350) is a “heavy” oil because it has a high fraction of long-chain hydrocarbons. Finally, “Arabian Light” oil is a light crude oil from Saudi Arabia.

First of all, samples comprising micro-volumes of oils of approximately 5 μL are taken using a gas chromatography micro-syringe injected into the boat. Finally, the mass of the samples is weighed using a precision scale. Inert silica may be added if necessary.

The boat 3 containing the sample is then supplied to the automatic sampler which allows the boat to be inserted into the pyrolysis furnace 4 via the piston 2 (see FIG. 1).

The evolution of the furnace temperature is programmed in advance according to a cycle ranging from a temperature T1 of preferably between 25° C. and 35° C. to a final temperature T2 of, preferably but not exclusively, between 640° C. and 660° C. The temperature variation ramp used is preferably between 15 and 25° C./min (see FIG. 2).

When the temperature in the pyrolysis furnace increases, the hydrocarbon signal detected by the FID 6 is recorded.

The results obtained for oils are then processed using specific software to visualize and process the analysis results from the Rock-Eval method. The acquired data are then imported into the Rock-Eval simulated distillation module. Depending on the relative position of the hydrocarbon curve on the mass/temperature graph (f(T)=m), the latter is segmented into sub-areas or clearly deconvolved according to a mathematical method chosen by the user based on the experimental Antoine equation.

Each of the areas obtained then corresponds to a class of hydrocarbons having the same number of carbon atoms and then makes it possible to obtain an estimate of the distribution of these hydrocarbons according to their number of carbon atoms (see FIG. 4).

Finally, knowing the boiling points of the corresponding n-alkanes, it is possible to reconstruct the simulated distillation curves for each of the three oils analyzed (see FIG. 5). The average molar mass of all the oil can also be estimated.

The composition of the three oils determined using the Rock-Eval simulated distillation method as well as that obtained via gas chromatography analysis is represented by the diagrams in FIGS. 6 to 8.

If these three oils are compared, each of them has its own “pattern” in terms of the distribution of hydrocarbon classes which results directly from its composition. Reference oil 775199 has a bimodality for its composition with approximately two third-thirds of light hydrocarbons and a third of heavier hydrocarbons, which results in a specific “pattern” of the hydrocarbon distribution diagram as well as of its simulated distillation curve. Reference oil 770350 is more homogeneous, which results in a Gaussian distribution of the proportions of the hydrocarbon component. The corresponding simulated distillation curve is of the sigmoidal type. Finally, Arabian Light has an asymmetrical Gaussian distribution centered on the C13 hydrocarbon but with varying proportions of heavy hydrocarbons that decrease as the hydrocarbon chains lengthen.

In order to be able to compare the simulated distillation results, respectively by Rock-Eval and by gas chromatography, normalization is necessary over the C1-C40 interval (characterization limit of the chromatography used) compared to the Rock-Eval data. As the Rock-Eval analysis allows the temperature to be increased up to 650° C. in the pyrolysis furnace, the entire quantity of oil is quantified unlike conventional chromatography analyses.

When comparing the data from simulated distillation, on the one hand by Rock-Eval and on the other hand by chromatography, it is observed that the “fingerprint” of the analyzed oil, which is materialized by its hydrocarbon distribution diagram and its simulated distillation curve, is largely preserved. A few differences nevertheless remain, all relative to the specificities of the two methods used.

Since the device is not provided with a separation column, a phenomenon of mixing of the analyzed hydrocarbons occurs. This is why the isomers far from the “average” isomer of the mixture (i.e., the most represented within the mixture) have affected boiling points and are moved towards the average temperatures of the mixture. This phenomenon is particularly well observed on the Rock-Eval distribution histogram of reference oil 775199 and in particular on the second peak centered around the C21 hydrocarbon while the latter is centered around the C26 isomers in the chromatography distribution diagram.

A further limit of the method of the invention results from the minimum starting temperature of the analysis cycle, which corresponds to the ambient temperature of the laboratory plus approximately 10° C., i.e., 30° C. This temperature does not allow hydrocarbons having fewer than 7 carbon atoms to be characterized. However, the method for characterizing C1-C6 hydrocarbons previously described in patent EP3918321 can complement the characterization of light hydrocarbons.

1. A method for the continuous characterization and quantification of C1-C120 hydrocarbons present in a single sample taken from a sedimentary basin, said method comprising pyrolysis under a non-oxidizing atmosphere of the sample, the entrainment of the gaseous pyrolysis effluents by means of an inert gas stream and then the analysis of said effluents in order to obtain their profiles as a function of the pyrolysis temperature, wherein said profiles are deconvolved into sub-areas corresponding to each class of isomer via a reference chart established via the equation (F) below for each of said classes and obtained using the empirical Antoine equation derived from the integration of the Clausius-Clapeyron physical law for an assumed constant enthalpy of vaporization;
mᵢ=(Dᵥ×Mᵢ×t)/(R×T)×10^{A-B/(T+C)}
with:
mᵢ (kg): mass of the hydrocarbon considered
Dᵥ (m³.s⁻¹): volume flow rate of the carrier gas
Mᵢ (kg.mol⁻¹): molar mass of the hydrocarbon considered
T (K): temperature
t (s): time
R (J.mol⁻¹.K⁻¹): ideal gas constant
A (Pa), B (-) and C (K): parameters of the Antoine equation
Pᵢ (Pa): partial pressure.

2. The method according to claim 1, wherein pyrolysis is carried out with a constant heating rate of between 0.5° C./min and 50° C./min in order to achieve a final temperature of between 30° C. and 900° C.

3. The method according to claim 1, wherein pyrolysis is carried out under a non-oxidizing atmosphere by means of inert gas flushing.

4. The method according to claim 1, wherein the effluents are analyzed by means of a flame ionization detector.

5. The method according to claim 1, wherein the sample is chosen from a group comprising source rocks, kerogen, coal, reservoir rocks, crude oils and heavy petroleum fractions, resins, asphaltenes, catalysts and soil samples.
